(19) United States
(12) Patent Application Publication
(10) Pub. No.: US 2010/0322987 A1
(43) Pub. Date: Dec. 23, 2010

(54) DENTAL WIPE

(75) Inventors: Richard Scott Robinson, Belle Mead, NJ (US); Richard J. Sullivan, Atlantic Highlands, NJ (US); Diane Cummins, Livingston, NJ (US)

Correspondence Address: COLGATE-PALMOLIVE COMPANY, 909 RIVER ROAD, PISCATAWAY, NJ, 08855, US

(73) Assignee: Colgate-Palmolive Company, New York, NY (US)

(21) Appl. No.: 12/866,764

(22) PCT Filed: Feb. 6, 2009
(86) PCT No.: PCT/US09/33297

§ 371 Date: Aug. 9, 2010

Related U.S. Application Data
(60) Provisional application No. 61/027,426, filed on Feb. 8, 2008.

(51) Int. Cl. A61K 8/02; A61K 8/18; A61Q 11/00

(52) U.S. Cl. 424/401; 424/49

(57) ABSTRACT

The present invention is directed to a dental wipe comprising a basic amino acid or salt thereof.

This application claims the benefit of U.S. Ser. No. 61/027,426 filed Feb. 8, 2008 the contents of which are incorporated herein by reference.

### BACKGROUND OF THE INVENTION

Dental wipes are known in the art, see e.g., U.S. Pat. No. 7,127,771, and are frequently used by individuals when conventional methods of cleaning of teeth and/or the oral cavity is inconvenient. For example, travelers and office workers may feel the need to clean their oral cavity, but brushing their teeth or using a mouthwash is inconvenient, or impossible. Parents of infants may desire to clean the infant's oral cavity, but use of a toothbrush or mouth wash is dangerous, as the infant may be injured by the brush, or swallow the mouthwash. Similarly, cleaning the oral cavity of invalids, e.g., hospitalized, unconscious people, is extremely difficult, but may be accomplished with dental wipes.

In view of the utility of dental wipes, there is a continuing need to develop more efficient and effective dental wipes.

### SUMMARY OF THE INVENTION

The present invention includes a dental wipe in combination or association with, e.g., impregnated, containing, or coated with, a composition (Composition 1.0) comprising a basic amino acid. The basic amino acid or salt may be coated onto the wipe, or impregnated within the wipe's matrix.

The invention thus includes dental wipes in combination or association with the following Compositions:
1.1 Composition 1.0 wherein the basic amino acid is arginine, lysine, citrullene, ornithine, creatine, histidine, diaminobutanoic acid, diaminoproprionic acid, salts thereof and/or combinations thereof.
1.2 Composition 1.0 or 1.0.1 therein the basis amino a
1.3 Any of the preceding compositions is provided in the form of a salt of a di- or tri-peptide comprising the basic amino acid.
1.4 Any of the preceding compositions wherein the basic amino acid is arginine.
1.5 Any of the preceding compositions wherein the basic amino acid is L-arginine.
1.6 Any of the preceding compositions wherein the basic amino acid is in salt form.
1.7 Any of the preceding compositions wherein the salt of the basic amino acid is arginine phosphate.
1.8 Any of the preceding compositions wherein the salt of the basic amino acid is arginine hydrochloride.
1.9 Any of the preceding compositions wherein the salt of the basic amino acid is arginine sulfate.
1.10 Any of the preceding compositions wherein the salt of the basic amino acid is arginine bicarbonate.
1.11 Any of the preceding compositions further comprising a fluoride salt selected from stannous fluoride, sodium fluoride, potassium fluoride, sodium monofluorophosphate, sodium fluorosilicate, ammonium fluorosilicate, amine fluoride, ammonium fluoride, and combinations thereof.
1.12 Any of the preceding compositions further comprising a physiologically acceptable potassium salt, e.g., potassium nitrate or potassium chloride, in an amount effective to reduce dentinal sensitivity.
1.13 Any of the preceding compositions comprising at least one humectant.
1.14 Any of the preceding compositions comprising at least one humectant selected from glycerin, sorbitol and combinations thereof.
1.15 Any of the preceding compositions comprising flavoring, fragrance and/or coloring.
1.16 Any of the preceding compositions comprising an antibacterial agent.
1.17 Any of the preceding compositions comprising an antibacterial agent selected from triclosan, herbal extracts and essential oils (e.g., rosemary extract, tea extract, magnolia extract, thymol, menthol, eucalyptol, geraniol, carvacrol, citral, hinokitol, catechol, methyl salicylate, epigallioeatechin gallate, epigallocatechin, gallic acid), bisguanide antiseptics e.g., chlorhexidine, alexidine or octenidine), quaternary ammonium compounds cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc citrate, stannous salts, copper salts, iron salts), sanguinarine, propolis and oxygenating agents (e.g., hydrogen peroxide, buffered sodium peroxyhorate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing.
1.18 Any of the preceding compositions further comprising an agent that interferes with or prevents bacterial attachment to a tooth or the oral cavity.
1.19 Any of the preceding compositions comprising a whitening agent.
1.20 Any of the preceding compositions comprising a whitening agent selected from a whitening active selected from the group consisting of peroxides, metal chlorites, perborates, percarbonates, peroxyacids, hypochlorites, and combinations thereof
1.21 Any of the preceding compositions further comprising hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate), or hydrogen peroxide polymer complexes such as hydrogen peroxide-polyvinyl pyrrolidone polymer complexes.
1.22 Any of the preceding compositions further comprising an anti-calculus agent.
1.23 Any of the preceding compositions further comprising an anti-calculus agent which is a polyphosphate. e.g., pyrophosphate, tripolyphosphate, or hexametaphosphate, e.g., in sodium salt form.
1.24 Any of the preceding compositions further comprising a source of calcium and phosphate selected from (i) calcium-glass complexes, e.g., calcium sodium phosphosilicates, and (ii) calcium-protein complexes, e.g., casein phosphopeptide-amorphous calcium phosphate.
1.25 Any of the preceding compositions further comprising a soluble calcium salt, e.g., calcium organophosphates or polyphosphates, e.g., calcium phytates, calcium glycerophosphate; salts of soluble carboxylic acids, e.g., selected from calcium citrate, calcium malate, calcium lactate, calcium acetate, calcium formate, calcium fumarate, calcium gluconate, calcium lactate gluconate, calcium aspartate, and calcium propionate; calcium chloride, calcium sulfate, calcium chloride, calcium nitrate; and mixtures thereof.
1.26 Any of the preceding compositions further comprising a surfactant, e.g., selected from anionic surfactants, e.g., sodium lauryl sulfate, and amphoteric surfactants, e.g., cocamidopropylbetaine, and mixtures thereof.
1.27 Any of the preceding compositions further comprising a breath freshener.
1.28 Any of the preceding compositions effective upon application to the oral cavity, e.g., with wiping, to
   a. reduce or inhibit formation of dental caries,
   b. reduce, repair or inhibit early enamel lesions, e.g. reduce, repair or inhibit pre-carious lesions of the enamel, e.g., as detected by quantitative light-induced fluorescence (QLF) or electrical conductance measurement (ECM),
   c. reduce or inhibit demineralization and promote remineralization of the teeth,
   d. reduce hypersensitivity of the teeth,
   e. reduce or inhibit gingivitis,
   f. promote healing of sores or cuts in the mouth,
   g. reduce levels of acid producing bacteria,
   h. to increase relative levels of arginolytic bacteria,
   i. inhibit microbial biofilm formation in the oral cavity,
   j. raise and/or maintain plaque pH at levels of at least pH 5.5 following sugar challenge,
   k. reduce plaque accumulation,
   l. treat dry mouth,
   m. whiten teeth,
   n. enhance systemic including cardiovascular health, by reducing potential for systemic infection via the oral tissues,
   o. reduce erosion of the teeth,
   p. immunize the teeth against cariogenic bacteria, and/or
   q. clean the teeth and oral cavity.
1.29 The present invention also encompasses method 2.0, a method to
   a. reduce or inhibit formation of dental caries,
   b. reduce, repair or inhibit early enamel lesions, e.g. reduce, repair or inhibit pre-carious lesions of the enamel, e.g., as detected by quantitative light-induced fluorescence (QLF) or electrical conductance measurement (ECM),
   c. reduce or inhibit demineralization and promote remineralization of the teeth,
   d. reduce hypersensitivity of the teeth,
   e. reduce or inhibit gingivitis,
   f. promote healing of sores or cuts in the mouth,
   g. reduce levels of acid producing bacteria,
   h. to increase relative levels of arginolytic bacteria,
   i. inhibit microbial biofilm formation in the oral cavity,
   j. raise and/or maintain plaque pH at levels of at least pH 5.5 following sugar challenge,
   k. reduce plaque accumulation,
   l. treat dry mouth,
   m. whiten teeth,
   n. enhance systemic health, including cardiovascular health, e.g., by reducing potential for systemic infection via the oral tissues,
   o. reduce erosion of the teeth,
   p. immunize the teeth against cariogenic bacteria, and/or
   q. clean the teeth and oral cavity.
comprising applying the Composition of the Invention to the oral cavity using a dental wipe.

Other embodiments of present invention will be apparent to one of skill in the

### DETAILED DESCRIPTION OF THE INVENTION

Without intending to be bound by a particular theory, it is believed that basic amino acids in the oral cavity are metabolized by certain types of bacteria, e.g., S. sanguis which are not cariogenic and which compete with cariogenic bacteria such as S. nutans, for position on the teeth and in the oral cavity. The arginolytic bacteria can use arginine and other basic amino acids to produce ammonia, thereby raising the pH of their environment, while cariogenic bacteria metabolize sugar to produce lactic acid, which tends to lower the plaque pH and demineralize the teeth, ultimately leading to cavities. It is believed that use of a Composition of the Invention may lead to a relative increase in the arginolytic bacteria and a relative decrease in the cariogenic bacteria, resulting in a higher plaque pH.

The basic amino acids which can be used in the compositions of the present the invention include not only naturally occurring basic amino acids, such as arginine, lysine, and histidine, but also any basic amino acids having a carboxyl group and an amino group in the molecule. Accordingly, basic amino acids include, but are not limited to, arginine, lysine, citrullene, ornithine, creatine, histidine, diaminobutanoic acid, diaminoproprionic acid, salts thereof or combinations thereof. In a particular embodiment, the basic amino acids are selected from arginine, citrullene, and ornithine, preferably, arginine, for example, 1-arginine.

The compositions of the invention are used in the mouth; salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form in a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion. A preferred salt is a bicarbonate, arginine bicarbonate.

Dental wipes and their methods of manufacture are well known in the art. For example, dental wipes may he produced by shaping non-woven materials so that it a finger may be inserted therein. See e.g., U.S. Pat. No. 6,721,987, the contents of which are herein incorporated by reference.

In one embodiment, a basic amino acid or salt thereof may he incorporated into the fibers used to produce the non-woven material.

Methods for coating woven and non-woven materials are also known in the art. Thus, in one embodiment, a basic amino acid or salt thereof may be sprayed directly on to the dental wipe. In one embodiment of the present invention, the dental wipe is treated in an emulsion bath comprising arginine or a pharmaceutically acceptable salt thereof, and then dried.

In one embodiment of the present invention, the dental wipe may optionally include fluoride, or a fluoride ion source. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., incorporated herein by reference. Representative fluoride ion sources include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof.

The dental wipe of the present invention may also comprise an antiseptic or antimicrobial, surfactant, whitening agent, calcium source, fluoride source, or other functional agents, e.g., as described above, and combinations thereof to further aid in the beneficial effects of the basic amino acid.

The compositions and methods according to the invention are useful to a method to protect the teeth by facilitating repair and remineralization, in particular to reduce or inhibit formation of dental caries, reduce or inhibit demineralization and promote remineralization of the teeth, reduce hypersensitivity of the teeth, and reduce, repair or inhibit early enamel lesions, e.g., as detected by quantitative light-induced fluorescence (QLF) or electronic caries monitor (ECM).

Quantitative Light-induced Fluorescence is a visible light fluorescence that can detect early lesions and longitudinally monitor the progression or regression. Normal teeth fluoresce in visible light; demineralized teeth do not or do so only to a lesser degree. The area of demineralization can be quantified and its progress monitored. Blue laser light is used to make the teeth auto fluoresce. Areas that have lost mineral have lower fluorescence and appear darker in comparison to a sound tooth surface. Software is used to quantify the fluorescence from a white spot or the area/volume associated with the lesion. Generally, subjects with existing white spot lesions are recruited as panelists. The measurements are performed in vivo with real teeth. The lesion area/volume is measured at the beginning of the clinical. The reduction (improvement) in lesion area/volume is measured at the end of 6 months of product use. The data is often reported as a percent improvement versus baseline.

Electrical Caries Monitoring is a technique used to measure mineral content of the tooth based on electrical resistance. Electrical conductance measurement exploits the fact that the fluid-filled tubules exposed upon demineralization and erosion of the enamel conduct electricity. As a tooth loses mineral, it becomes less resistive to electrical current due to increased porosity. An increase in the conductance of the patient's teeth therefore may indicate demineralization. Generally, studies are conducted of root surfaces with an existing lesion. The measurements are performed in vivo with real teeth. Changes in electrical resistance before and after 6 month treatments are made. In addition, a classical caries score for root surfaces is made using a tactile probe. The hardness is classified on a three point scale: hard, leathery, or soft. In this type of study, typically the results are reported as electrical resistance (higher number is better) for the ECM measurements and an improvement in hardness of the lesion based on the tactile probe score.

The Compositions of the Invention are thus useful in a method to reduce early lesions of the enamel (as measured by QLF or ECM) relative to a composition lacking effective amounts of fluorine and/or arginine.

The Compositions of the invention are additionally useful in methods to reduce harmful bacteria in the oral cavity, for example methods to reduce or inhibit gingivitis, reduce levels of acid producing bacteria, to increase relative levels of arginolytic bacteria, inhibit microbial biofilm formation in the oral cavity, raise and/or maintain plaque pH at levels of at least pH 5.5 following sugar challenge, reduce plaque accumulation, and/or clean the teeth and oral cavity.

Finally, by increasing the pH in the mouth and discouraging pathogenic bacteria, the Compositions of the Invention are useful to promote healing of sores or cuts in the mouth.

Enhancing oral health also provides benefits in systemic health, as the oral tissues can be gateways for systemic infections. Good oral health is associated with systemic health, including cardiovascular health. The compositions and methods of the invention provide particular benefits because basic amino acids, especially arginine, are sources of nitrogen which supply NO synthesis pathways and thus enhance microcirculation in the oral tissues. Providing a less acidic oral environment is also helpful in reducing gastric distress and creates an environment less favorable to Heliobacter, which is associated with gastric ulcers. Arginine in particular is required for high expression of specific immune cell receptors, for example T-cell receptors, so that arginine can enhance an effective immune response. The compositions and methods of the invention are thus useful to enhance systemic health, including cardiovascular health.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. It is understood that when formulations are described, they may be described in terms of their ingredients, as is common in the art, notwithstanding that these ingredients may react with one another in the actual formulation as it is made, stored and used, and such products are intended to be covered by the formulations described.

The following examples further describe and demonstrate illustrative embodiments within the scope of the present invention. The examples are given solely for illustration and are not to be construed as limitations of this invention as many variations are possible without departing from the spirit and scope thereof. Various modifications of the invention in addition to those shown and described herein should be apparent to those skilled in the art and are intended to fall within the appended claims.

1. A dental wipe comprising a non-woven material; and a composition comprising a basic amino acid.

2. The dental wipe of claim 1 wherein the basic amino acid is selected from arginine, lysine, citrullene, ornithine, creatine, histidine, diaminobutanoic acid, diaminoproprionic acid, salts thereof, and combinations thereof.

3. The dental wipe of claim 1 wherein the basic amino acid has the L-configuration.

4. The dental wipe of claim 1 wherein the basic amino acid is provided in the form of a salt of a di- or tri-peptide comprising the basic amino acid.

5. The dental wipe of claim 1 wherein the basic amino acid is arginine.

6. The dental wipe of claim 3 wherein the basic amino acid is L-arginine.

7. The dental wipe of claim 1 wherein the basic amino acid is in salt form.

8. The dental wipe of claim 7 wherein the basic amino acid is arginine, and the salt form is selected from arginine phosphate, arginine hydrochloride, arginine sulfate, arginine bicarbonate and combinations thereof.

9. The dental wipe of claim 1 wherein the composition further comprises a fluoride ion source selected from the group consisting of stannous fluoride, sodium fluoride, potassium fluoride, sodium monofluorophosphate, sodium fluorosilicate, ammonium fluorosilicate, amine fluoride, ammonium fluoride, and combinations thereof.

10. The dental wipe of claim 1 wherein the composition further comprises at least one humectant selected from the group consisting of glycerin, sorbitol and combinations thereof.

11. The dental wipe of claim 1 wherein the composition further comprises flavoring, fragrance and/or coloring.

12. The dental wipe of claim 1 wherein the composition further comprises an antibacterial agent selected from triclosan, herbal extracts and essential oils, bisguanide antiseptics, quaternary ammonium compounds, phenolic antiseptics, hexetidine, povidone iodine, delmopinol, salifluor, metal ions, sanguinarine, propolis and combinations thereof.

13. The dental wipe of claim 1 wherein the composition further comprises an agent that interferes with or prevents bacterial attachment to a tooth or the oral cavity.

14. A method to:
a. reduce or inhibit formation of dental caries,
b. reduce, repair or inhibit early enamel lesions, e.g. reduce, repair or inhibit pre-carious lesions of the enamel, e.g., as detected by quantitative light-induced fluorescence (QLF) or electrical conductance measurement (ECM),
c. reduce or inhibit demineralization and promote remineralization of the teeth,
d. reduce hypersensitivity of the teeth,
e. reduce or inhibit gingivitis,
f. promote healing of sores or cuts in the mouth,
g. reduce levels of acid producing bacteria,
h. increase relative levels of arginolytic bacteria,
i. inhibit microbial biofilm formation in the oral cavity,
j. raise and/or maintain plaque pH at levels of at least pH 5.5 following sugar challenge,
k. reduce plaque accumulation,
l. treat dry mouth,
m. whiten teeth,
n. promote systemic health, including cardiovascular health, e.g., by reducing potential for systemic infection via the oral tissues,
o. reduce erosion of the teeth,
p. immunize the teeth against cariogenic bacteria, and/or
q. clean the teeth and oral cavity;
comprising swabbing the oral cavity of a subject a with the dental wipe according to claim 1.

15-17. (canceled)

18. The dental wipe of claim 1, wherein the composition further comprises a potassium ion source.

19. The dental wipe of claim 18, wherein said potassium ion source is selected from potassium nitrate and potassium chloride.
